(19) United States
(12) Patent Application Publication
(10) Pub. No.: US 2010/0022970 A1
(43) Pub. Date: Jan. 28, 2010

(54) COLLECTING SHEATH AND METHOD OF USE THEREOF

(76) Inventor: Eran Hirszowicz, Ramat-Gan (IL)

Correspondence Address: DANIEL J SWIRSKY, 55 REUVEN ST., BEIT SHEMESH, 99544, IL

(21) Appl. No.: 12/306,933

(22) PCT Filed: Jul. 5, 2007
(86) PCT No.: PCT/IL07/00844

§ 371 Date: Sep. 13, 2009

Related U.S. Application Data
(60) Provisional application No. 60/818,528, filed on Jul. 6, 2006.

(51) Int. Cl. A61M 25/00

(52) U.S. Cl. 604/268

(57) ABSTRACT

The present invention is directed to a device and method for safely removing matter collected by matter collecting catheter from a body organ or passage by means of a collecting sheath comprising an elongated tube having at its distal end an expandable container sleeve, and a lumen passing along said elongated tube and said expandable container sleeve, wherein said lumen is suitable for passage of the matter collecting catheter therethrough. The expandable container sleeve may be introduced into the body organ or passage in a folded state for reducing its cross-sectional area.

### FIELD OF THE INVENTION

The present invention relates to means for removing matter from a body organ or internal passage. More particularly, the invention relates to a device and method for safely removing matter collecting catheter having collected matter gathered from a body organ or internal passage.

### BACKGROUND OF THE INVENTION

Balloon catheters capable of collecting matter (e.g., debris) from a body organ or passage way (e.g., blood vessel) are described in detail in International Patent Applications Serial Nos. WO 2007/004221 and WO 2007/042935 both entitled “balloon catheter”, and in U.S. patent application Ser. No. 11/477,812 entitled “balloon catheter”, filed on Jun. 30, 2006, all of which are of the present assignee, the entire disclosures of which are incorporated herein by reference.

For the sake of clarity the structure and operation of a possible embodiment of balloon catheter 10 (hereinafter also referred to as matter collecting catheter) designed to collect matter 15 from a body organ or internal passage 12 will be briefly described with reference to FIG. 1. Balloon catheter 10 comprises an outer tube 18 containing an inner tube 19 passing thereinside such that a distal end thereof outwardly protrudes via the distal opening of outer tube 18. Balloon 5 may be implemented by an inflatable sleeve having tapering ends configured to tightly fit over tubes 18 and 19 in attachments 7 and 6, respectively. This tube structure defines an annular inflation lumen 16 between tubes 18 and 19 which may be used to inflate balloon 5 with suitable inflation media. As shown in FIG. 1 balloon 5 may be folded inwardly by pulling inner tube 19 proximally and thereafter discharging portions of inflation media from balloon 5, which in turn forms a cavity 14 in the folded balloon structure, that may be advantageously used to collect matter 15 (e.g., debris) from body organ or internal passage 12 (e.g., blood vessel).

However, after collecting matter 15 in balloon 5 the deflated diameter of balloon 5 in its inwardly folded state may be greater than the diameter of the inner lumen of a guiding catheter (30, shown in FIG. 3) through which it should be removed from the body of the treated subject. If balloon 5 is proximally pulled into the guiding catheter it will be squeezed and some of the collected material 15 may be discharged from cavity 14 back into body organ or internal passage 12.

A collection device is described in WO 05/112783, wherein a barrier membrane connected to an expandable clamp is used for collecting a tissue sample. After collecting the tissue the clamp is retracted into the collection device such that the barrier membrane connected thereto is changed into a pouch configuration containing the sample tissue.

A modular adjustable blood filter device is described in U.S. Pat. No. 6,152,947. This filter apparatus includes a shaft and a filter frame adjustable between a contracted condition and an enlarged condition, a frame sizing mechanism and a filter mesh coupled thereto. After insertion the frame sizing mechanism adjusts the diameter of the filter frame to conform to the inner lumen of the vessel. The adjustable frame may be closed into a contracted state for entrapping embolic material, and the adjustable filter may be withdrawn into an insertion device.

Another emboli capturing system is described in US 2005/0101986, wherein an expandable emboli capturing device is mounted near the distal end of an elongate member, and is movable between a radially expanded position, in which the emboli capturing device forms a basket with a proximally opening mouth, and a radially contracted position. A delivery device having a receiving end is configured to receive the emboli capturing device and retains at least the mouth of the emboli capturing device in a radially retracted position.

The clot removal device described in WO 02/055146 includes a proximal obstructive matter retaining member having a radially flared and splayable distal end capable of being advanced out of a distal end of a guide catheter such that the clot is captured between the distal obstructive matter receiving portion and the flared distal end of the proximal obstructive matter retaining member.

In US 2002/0121472 an intravascular filter is used for collecting debris dislodged during a medical procedure, wherein an actuatable dilator tip is utilized to retrieve the filter at least in part within a distal sheath. US 2002/0082639 describes an emboli capturing system which includes an expandable member having spaced portion spaced radially outwardly from a guide-wire, and emboli capturing device being coupled to an expandable member.

There is still a need for suitable means for safely removing matter collecting catheter having collected matter gathered from body organ or internal passage.

It is therefore an object of the present invention to provide a device and method for removing matter collected from a body organ or internal passage.

It is another object of the present invention to provide a device and method for holding, and preventing the discharge of, matter collected by a matter collecting catheter form a body organ or internal passage.

Other objects and advantages of the invention will become apparent as the description proceeds.

### SUMMARY OF THE INVENTION

The present invention is directed to a device and method for safely removing matter collected by a matter collecting catheter from a body organ or passage. The inventor of the present invention found out that matter collected by means of a matter collecting catheter from a body organ or passage can be safely removed from the body of the treated subject by means of a collecting sheath comprising an elongated tube having at its distal end an expandable container sleeve, and a lumen passing along said elongated tube and said expandable container sleeve, wherein said lumen is suitable for passage of the matter collecting catheter therethrough.

The expandable container sleeve may be introduced into the body organ or passage in a folded state for reducing its cross-sectional area. The matter collecting catheter may be introduced before or after introducing the collecting sheath into the body organ or passage, or alternatively it may be contained in its lumen during insertion thereof.

The collecting sheath may be introduced into the body organ or passage over a guidewire, over a previously introduced matter collecting catheter, or via a guiding catheter.

After collecting material from the body organ or passage by the material collecting catheter, it is introduced via a distal opening in expandable container sleeve into the lumen of the collecting sheath, thereby forcing said expandable container sleeve to radially expand and open into an unfolded state. Thereafter, the collecting sheath and the material collecting catheter contained in its lumen are retracted proximally and removed from the body of the treated subject.

In one aspect the present invention is directed to a device for removing matter collected by a matter collecting catheter from a body organ or passage, said device comprises an elongated tube having at a distal end thereof an expandable container sleeve, and a lumen passing therealong, wherein said lumen is suitable for the passage of said matter collecting catheter therethrough, and wherein said expandable container sleeve is introduced into the body organ or passage in a folded state, and wherein said expandable container sleeve is capable of being expanded radially into an unfolded state by introducing into its lumen via its distal end opening a distal portion of said matter collecting catheter with matter collected thereby.

Preferably, the expandable container sleeve includes in its folded state numerous corrugations formed along its length, said corrugations may form reentrant overlapping folds (folding rings, annularbellow folds).

In a specific embodiment of the invention a balloon catheter device is employed for collecting matter from the body organ or passage by means of an invaginated balloon. Advantageously, the length of expandable container sleeve is slightly greater than half of the length of the balloon in its invaginated state.

The balloon catheter may comprise sleeve fastening means for fastening the distal edge of expandable container sleeve for preventing inadvertent opening thereof and maintaining it in its folded state. Said sleeve fastening means may be configured in a shape of a skirt having a narrow distal end portion fitted over the outer surface of balloon catheter, and a wider proximal end portion configured to receive and secure the distal edge of expandable container sleeve when in its folded state. Advantageously, expandable container sleeve may be manufactured from a resilient material (e.g., silk, nylon, PET).

In another aspect the present invention is directed to a method for removing material collected by a matter collecting catheter from a body organ or passage, the method comprising: introducing into said body organ or passage a device comprising an elongated tube having at its distal end an expandable container sleeve, and a lumen passing therethrough, wherein said lumen is suitable for the passage of said matter collecting catheter through said elongated tube and said expandable container sleeve, and wherein said expandable container sleeve is introduced into the body organ or passage in a folded state; introducing into the lumen passing through said expandable container sleeve via a distal opening thereof a distal portion of said matter collecting catheter having matter collected thereby, thereby radially expanding it into its unfolded state; removing said device and the matter collecting catheter contained therein from the body of the treated subject.

The device may be introduced into the body organ or passage before or after insertion of the matter collecting catheter into the body organ or passage. The device may be introduced into the body organ or passage over a guidewire, over the matter collecting catheter, or via a guiding catheter.

The matter collecting catheter may be implemented by a balloon catheter device capable of collecting matter from the body organ or passage by means of an invaginated balloon. The balloon catheter may comprise sleeve fastening means for fastening the distal edge of expandable container sleeve, wherein the state of said expandable container sleeve is changed into its unfolded state by releasing said fastening of said sleeve fastening means.

### BRIEF DESCRIPTION OF THE DRAWINGS

The present invention is illustrated by way of example in the accompanying drawings, in which similar references consistently indicate similar elements and in which:

FIG. 1 schematically illustrates a possible implementation of a balloon catheter suitable for collecting matter from a body organ or internal passage;

FIG. 2 schematically illustrates a preferred embodiment of collecting sheath device of the invention;

FIG. 3 schematically illustrates introducing the balloon catheter and the collecting sheath device via a guiding catheter;

FIG. 4 schematically illustrates the balloon catheter and collecting sheath device after collecting matter by the balloon;

FIG. 5 schematically illustrates inserting the balloon catheter into a container sleeve of the collecting sheath; and

FIGS. 6A and 6B schematically illustrate a preferred embodiment of the collecting sheath and balloon catheter of the invention wherein the outer shaft of the balloon catheter comprises sleeve fastening means.

### DETAILED DESCRIPTION OF PREFERRED EMBODIMENTS

The present invention is directed to a device and method for safely removing collected matter from a body organ or internal passage. While the collected matter may be obtained in various ways, in the preferred embodiment of the invention it is sucked into a cavity formed in an invaginated balloon of a balloon catheter device by folding a distal portion thereof inwardly as exemplified hereinabove with reference to FIG. 1. The device of the invention is designed to withstand forces during retraction which squeeze the inwardly folded balloon containing the collected matter via a relatively narrow guiding catheter without discharging collected matter back into the treatment site or elsewhere in the body.

In general, the matter removing device (also referred to herein as collecting sheath) of the invention comprises an elongated tube, designed to allow passage of the balloon catheter therethrough, and a container sleeve connected to the distal end opening of said elongated tube. The collecting sheath containing the balloon catheter is preferably introduced into the body of the treated subject via a guiding catheter in a state in which its container sleeve is folded to assume a reduced cross-sectional profile. After reaching the treatment site the distal part of the balloon catheter containing the balloon is advanced outwardly via the distal opening of container sleeve for performing the requisite treatment and collecting matter obtained therein. The container sleeve may be then opened into its unfolded state by retracting the distal part of balloon catheter proximally thereinto, such that the inwardly folded balloon is pressed thereinto and thus radially push the wall of folded container sleeve into an open, unfolded state.

Alternatively, the balloon catheter is introduced first e.g., over a guidewire, and the collecting sheath is advanced thereover, before or after carrying the requisite treatment and collecting matter with the balloon catheter.

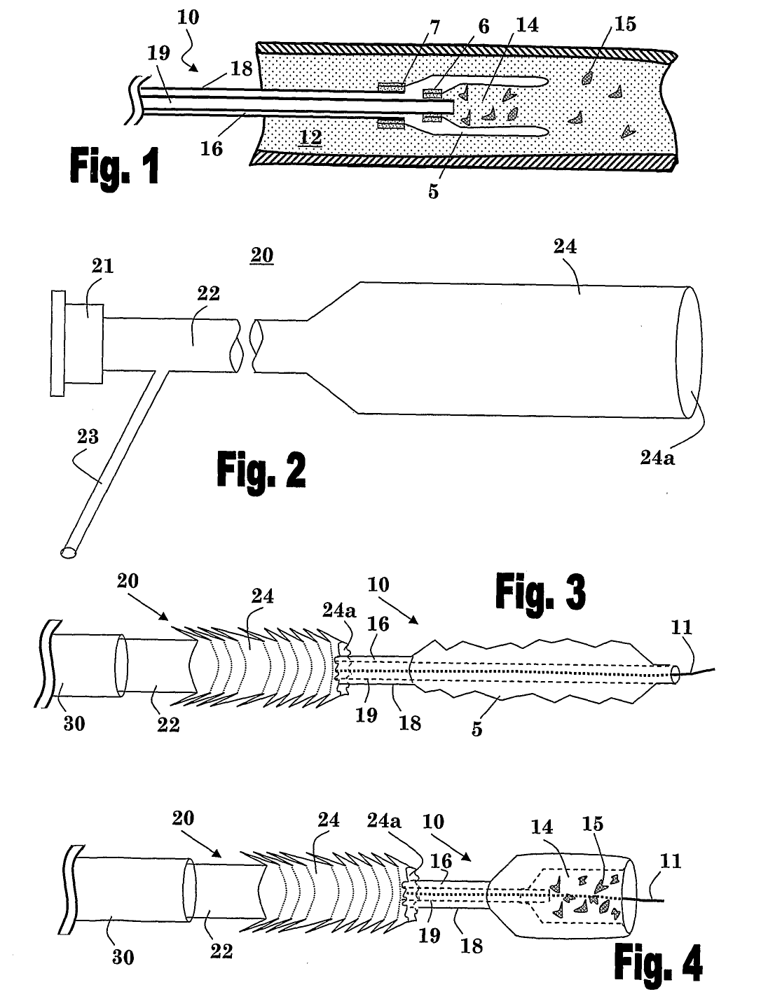

FIG. 2 schematically illustrates a preferred embodiment of a collecting sheath 20 of the invention which may be used to remove matter collected from a body organ or internal passage. Collection sheath 20 comprises a long tube 22 the inner lumen of which is of a diameter that permits it to be threaded over balloon catheter 10 (shown if FIGS. 3 to 5), and a container sleeve 24, configured to receive the balloon catheter 10 after the collection of matter from the treatment site and safely remove it therefrom through the guiding catheter (30) out of the body of the treated subject. The length of container sleeve 24 should be longer than the length of balloon 5 when in its folded state (shown in FIG. 1).

The proximal end of tube 22 of collection sheath 20 comprises a proximal seal 21 which is used to prevent outflow of fluids (e.g., blood) therefrom during the treatment procedure, or of flushing fluids obtained therein when the device is flushed via optional flushing tube 23. Optional flushing tube 23 is connected to tube 22 and allows repeated flushing of the sheath by suitable fluids (e.g., heparinized saline), before or after treatment.

FIG. 3 schematically illustrates introducing the balloon catheter 10 and collecting sheath 20 into the treatment site (not shown) via a guiding catheter 30. The collecting sheath 20 may be advanced through guiding catheter 30 towards the treatment site while balloon catheter 10 is contained in its lumen. Alternatively, collecting sheath 20 may be kept proximally ready for insertion into the treatment site allowing insertion of balloon catheter therethrough, such that it may be threaded over balloon catheter 10 in a later stage of the procedure. As exemplified in FIG. 3, during insertion into the body of the treated subject container sleeve 24 is in a folded state, to permit easy advancement through guiding catheter 30; balloon 5 is similarly in a deflated state.

As exemplified in FIG. 3, container sleeve 24 may include in its folded state numerous corrugations formed along the length of container sleeve 24. Said corrugations may from reentrant overlapping folds (folding rings, annularbellow folds) such that a drinking straw configuration is obtained.

Generally, debris or other particulate matter is collected by balloon 5 by inflating it in the treatment site, pulling inner tube 19 proximally, to inwardly fold balloon 5, and thereafter discharging portions of the inflation media from balloon 5 to form cavity 14, into which matter 15 is gathered due to the suction effect obtained. Preferably, balloon 5 is completely deflated to a state in which negative pressure conditions are obtained in it and in annular inflation lumen 16. Guidewire 11, threaded through the lumen of inner tube 19, may be used to maneuver balloon catheter 10 in the body of the treated subject. FIG. 4 schematically illustrates balloon catheter 10 and collecting sheath 20 after collecting matter 15 from treatment site by balloon 5.

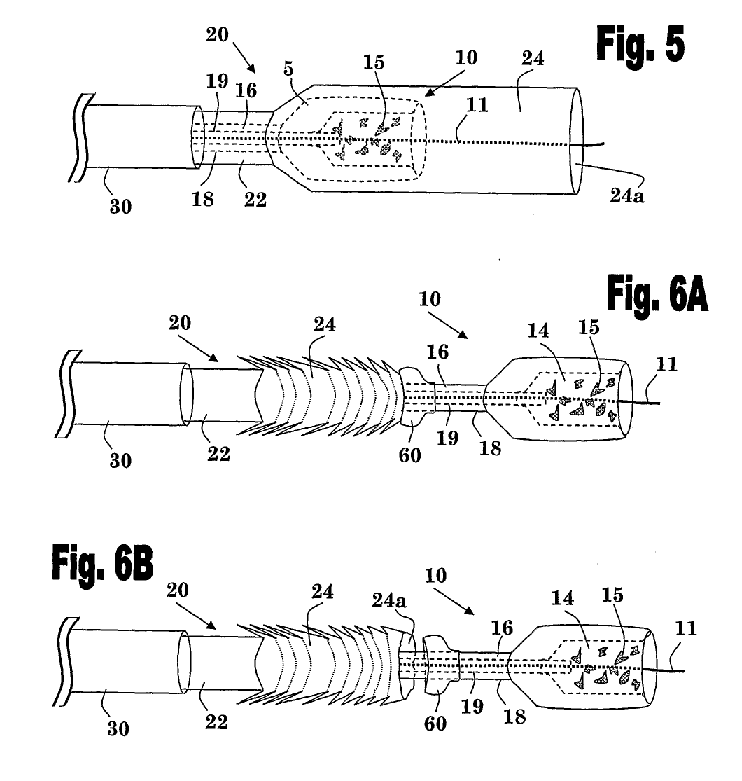

After collecting matter 15 by balloon 5, the operator may move balloon 5 and collection sheath 20 progressively closer to each other until balloon 5 enters through the distal end opening 24a into the internal cavity of container sleeve 24 which is thereby caused to open into its unfolded state, as shown in FIG. 5.

Thereafter, collecting sheath 20 and balloon catheter 10 are pulled backward (proximally)(not shown) into guiding catheter 30. In effect, container sleeve 24 and balloon 5 are squeezed into guiding catheter 30, during which a portion of the collected matter 15 may escape from cavity 14 into container sleeve 24. However, this portion of discharged matter remains in container sleeve 24 near the distal end of tube 22 of collection sheath 20. The collection sheath 20, comprising balloon catheter 10 and collected matter 15 inside it, is then pulled backward through guiding catheter 30 until it is safely removed from the body of the treated subject.

The main body of collection sheath 20 may be manufactured from any suitable biocompatible material such as nylon 12 or pebax or from a similar compound. Container sleeve 24 can be made from the same materials as the collection sheath 20 body, or from more complex materials comprising additives (e.g., glass fibers, stainless steel mesh, carbon fibers, etc.) which can reinforce it against buckling. The surfaces of collection sheath 20 may be treated, or suitable additives may be added, to the materials it is made from to reduce its surface friction. Some radio opaque markers may be added to the distal portion of collection sheath 20 to enhance imaging during the procedure.

The inner diameter of tube 22 should be compatible to the outer diameter of balloon catheter 10, which is about 0.8 mm to 1.5 mm. The wall thickness of collection sheath 20 may vary between 0.05 mm to 0.2 mm. The length of collection sheath 20 is preferably slightly shorter than the length of balloon catheter 10, which is about 0.7 meter to 1.5 meter.

The length of container sleeve 24 is generally between 2 mm to 10 mm longer than half of the length of balloon 5 in its unfolded state. The inner diameter of container sleeve 24 is preferably the same as, or slightly greater than, the outer diameter of the balloon 5 in its inflated state. The wall thickness of container sleeve 24 should be as small as possible in order to reduce its cross-section profile and still retain stability, for example, it may be in range of 5 micron to 50 micron.

In another preferred embodiment of the invention, shown in FIGS. 6A and 6B, sleeve fastening means 60 mounted over outer tube 18 is used for fastening the distal edge of container sleeve 24 and thereby preventing inadvertent opening thereof and maintaining it in its folded state. Sleeve fastening means 60 may be configured in a shape of a skirt having a narrow distal end portion, designed to tightly fit over the outer surface of outer tube 18, and a wider proximal end portion configured to receive the distal edge of container sleeve 24, when in its folded state, as shown in FIG. 6A.

After insertion of collecting sheath 20 and balloon catheter 10 into the treatment site, the balloon and the sleeve are moved apart, for example by advancing balloon catheter distally relative to collecting sheath 20 to release the fastening of sleeve fastening means 60, or alternatively, by retracting collecting sheath 20 proximally relative to balloon catheter 10. As demonstrated in FIG. 6B, after advancing balloon catheter 10 distally container sleeve 24 may start to open into its unfolded state. Advantageously, container sleeve 24 may be manufactured from a resilient material (e.g., silk, nylon, PET) such that after releasing the fastening of its distal edge its entire length will open into its unfolded state (shown in FIG. 2).

Sleeve fastening means 60 may be manufactured from a type of elastomer, such as polyurethane, pebax or silicone, preferably from polyurethane, and it may be attached to the outer surface of tube 18 by thermal bounding. Alternatively, sleeve fastening means 60 may be manufactured as an integral part of balloon catheter 10. The length of sleeve fastening means 60 may generally be in the range of 1 mm to 3 mm, preferably about 2 mm, and the diameter of its proximal end portion may generally be in the range of 1 mm to 3 mm, preferably about 1.5 mm.

In a preferred embodiment of the invention, the matter collecting device is used for collecting atherosclerotic debris from an intravascular lesion, such as may commonly be found in the coronary arteries.

All of the abovementioned parameters are given by way of example only, and may be changed in accordance with the differing requirements of the various embodiments of the present invention. Thus, the abovementioned parameters should not be construed as limiting the scope of the present invention in any way. In addition, it is to be appreciated that the different tubes, balloons, sleeves, and other members, described hereinabove may be constructed in different shapes (e.g. having oval, square etc. form in plan view) and sizes differing from those exemplified in the preceding description.

The above description has of course been provided only for the purpose of illustration, and is not intended to limit the invention in any way. As will be appreciated by the skilled person, the invention can be carried out in a great variety of ways, employing more than one technique from those described above, all without exceeding the scope of the invention.

1. A device for removing matter collected by a matter collecting catheter from a body organ or passage, comprising an elongated tube having at its distal end thereof an expandable container sleeve, and a lumen passing therethrough, wherein said lumen is suitable for the passage of said matter collecting catheter through said elongated tube and said expandable container sleeve, and wherein said expandable container sleeve is introduced into the body organ or passage in a folded state, and wherein said expandable container sleeve is expanded radially into an unfolded state by introducing thereinto a distal portion of said matter collecting catheter.

2. The device according to claim 1, wherein the expandable container sleeve includes in its folded state numerous corrugations formed along its length.

3. The device according to claim 1, wherein the matter collecting catheter is a balloon catheter device.

4. The device according to claim 3, wherein the balloon catheter device is capable of collecting matter from the body organ or passage by means of an invaginated balloon.

5. The device according to claim 4, wherein the length of expandable container sleeve is slightly greater than half of the length of the balloon in its invaginated state.

6. The device according to claim 3, wherein the balloon catheter comprises sleeve fastening means for fastening the distal edge of expandable container sleeve.

7. The device according to claim 6, wherein the sleeve fastening means is configured in a shape of a skirt having a narrow distal end portion and a wider proximal end portion configured to receive and secure the distal edge of expandable container sleeve in its folded state.

8. The device according to claim 1, wherein the expandable container sleeve is made from a resilient material.

9. The device according to claim 1, wherein the expandable container sleeve is made from one or more materials selected from the following group: silk, nylon, PET.

10. The device according to claim 1, wherein a guiding catheter is used for introducing/removing said device into/from the body of the treated subject.

11. A method for removing material collected by a matter collecting catheter from a body organ or passage, comprising: introducing into said body organ or passage a device comprising an elongated tube having at its distal end an expandable container sleeve, and a lumen passing therethrough, wherein said lumen is suitable for the passage of said matter collecting catheter through said elongated tube and said expandable container sleeve, and wherein said expandable container sleeve is introduced into the body organ or passage in a folded state; introducing into said expandable container sleeve a distal end portion of said matter collecting catheter, thereby radially expanding it into its unfolded state; removing said device and the matter collecting catheter contained in its lumen from the body of the treated subject.

12. The method according to claim 11, wherein the device is introduced into the body organ or passage before insertion of the matter collecting catheter into the body organ or passage.

13. The method according to claim 11, wherein the device is introduced into the body organ or passage after insertion of the matter collecting catheter into the body organ or passage.

14. The method according to claim 11, wherein the device is introduced into the body organ or passage over a guidewire or over the matter collecting catheter.

15. The method according to claim 11, wherein the device is introduced into the body organ or passage via a guiding catheter.

16. The method according to claim 11, wherein the matter collecting catheter is a balloon catheter device.

17. The method according to claim 16, wherein the balloon catheter device is capable of collecting matter from the body organ or passage by means of an invaginated balloon.

18. The method according to claim 16, wherein The balloon catheter comprises sleeve fastening means for fastening the distal edge of expandable container sleeve, wherein the state of said expandable container sleeve is changed into its unfolded state by releasing said fastening of said sleeve fastening means.
